(19) United States
(12) Patent Application Publication
(10) Pub. No.: US 2021/0346199 A1
(43) Pub. Date: Nov. 11, 2021

(54) INSTRUMENT FOR INTRAOCULAR SURGICAL PROCEDURE

(71) Applicant: GEUDER AG, Heidelbeng (DE)

(72) Inventors: Theodor Stappler, Clarens (DE); Juergen EDER, Gaiberg (DE); Hamadi EL-AYARI, Frankfurt (DE)

(21) Appl. No.: 17/277,950

(22) PCT Filed: Sep. 19, 2019
(86) PCT No.: PCT/DE2019/200111

§ 371 Date: Mar. 19, 2021

(30) Foreign Application Priority Data
Sep. 19, 2018 (DE) 10 2018 215 965.3

(51) Int. Cl. A61F 9/007

(52) U.S. Cl. CPC A61F 9/00736

(57) ABSTRACT

An instrument for intraocular surgical procedure comprises a handle, a probe designed as a hollow body connected to the handle, and a core arranged in the probe, with an active element at the end for removing impurities from the eye, wherein either the probe is displaceable at least partially out of the handle and into it with respect to the core firmly connected to the handle, or the core is slidable at least partially out of the handle and into it with respect to the probe firmly connected to the handle, so that the core with the active element is completely within the probe or protrudes from the probe by a predetermined length, with an actuating device for actuating the probe or core being provided in the handle.

This patent application is a 35 U.S.C. § 371 national stage entry based on and claiming priority to International patent application PCT/DE2019/200111, filed on Sep. 19, 2019, which in turn claims priority to German patent application DE 10 2018 215 965.3, filed on Sep. 19, 2018, the disclosures of which are incorporated by reference in their entirety.

The disclosure relates to an instrument for intraocular surgical procedure, specifically an intraocular cleaning instrument.

Instruments for intraocular surgical procedure are well known from the practice of ophthalmology. These are usually light guides used for lighting, knives of various designs, tweezers, vitrectors for core vitrectomy, etc. Most of these instruments have a trocar-capable probe, i.e. a probe that can be inserted through a trocar into the eye.

The instrument being discussed here is an ophthalmic surgical instrument with a handle, a probe designed as a hollow body connected to the handle, and a core arranged in the probe.

Contamination of any kind in the eye cannot be avoided during eye surgery. It is already known from practice to aspirate such impurities from the eye—the front section or the rear section of the eye. Due to adhesion, this is only possible to a limited extent.

The present disclosure is therefore based on the object of realizing an effective removal of “soiling” by means of an intraocular cleaning instrument.

For this purpose, the core of the instrument discussed above is equipped at its end, i.e. at its distal end, with an active element for removing impurities in the eye. Ultimately, the active element is a cleaning device in the broadest sense in order, for example, to wipe off and remove foreign bodies such as silicone droplets from the inside of the eye from a membrane or the like.

According to the disclosure, the active element is used for cleaning, namely to remove impurities in the eye. The instrument is equipped with a very special technology, namely that either the probe is displaceable at least partially out of the handle and into it with respect to the core firmly connected to the handle, or the core is slidable at least partially out of the handle and into it with respect to the probe firmly connected to the handle, so that the core with the active element is completely within the probe (in a rest position) or protrudes from the probe by a predetermined length (in a working position), with an actuating device for actuating the probe or core being provided in the handle.

It is essential for the disclosure that the core with the active element at the end can be displaced out of the probe in order to reach the respective site of the contamination within the eye. Two basic mechanisms are conceivable, namely to the effect that the core is at least partially pushed out of the probe or that the probe is pushed back or into the handle with the core fixed with respect to the handle. In both cases, the core with the active element at the end is exposed and can thus be brought to the respective site of action in the eye.

The active element is to be understood as a cleaning element, wherein the active element can specifically be designed as a brush, mop, cloth, sponge, small wiper blade, as a fibrous, flat, ball-like element, etc. It can also be advantageous if the active element is suitable for aspiring impurities, for example for aspiring silicone oil droplets resulting from surgery/treatment on and/or in the eye.

Depending on the type of intended surgery, the core can be designed substantially in a straight line in pushed-out state, at least when it is possible to reach the point to be cleaned, for example on the lens, in a straight line. If, on the other hand, an area in the posterior chamber of the eye is to be reached, it can be advantageous if the core is preferably bent by 45 degrees to 180 degrees towards the distal end so that, when the core is pushed out, the bend undergoes elastic relaxation of the core material and unfolds, and when the core is pushed in, it builds up elastic bias against the inner wall of the probe.

In other words, with the instrument according to the disclosure it is possible to first, with the core being inserted, insert the probe through a trocar and an incision and to unfold the core and the active element within the eye, which enables particularly gentle surgery.

The handle can be designed in the sense of a housing, wherein it can advantageously be designed as a sleeve tapering towards the distal end and/or towards the probe. An actuating element of an actuating device can be provided in this sleeve, the displacement of which allows the core to be exposed. The actuating element can be designed as an inner sleeve or bolt running inside the handle, wherein it preferably runs over a limited sliding distance. This defines the extent to which the core extends from the probe.

In a further advantageous manner, the actuating device comprises a locking mechanism for locking a relative position between the probe and the core. Specifically, this can be a clamping device, a locking screw, etc.

In a particularly advantageous manner, a reservoir for a liquid is formed in the handle, which can be discharged through the probe and, optionally, through the core towards the active element. This liquid can be a saline solution, for example. In any case, the liquid is used together with the active element for cleaning in the broadest sense.

Alternatively, it is conceivable that a connection for connecting or plugging-in a syringe is formed at the proximal end of the handle, the contents of which can be discharged through the handle and through the probe and, optionally, through the core towards the active element. By actuating the syringe, the liquid contained therein, specifically by actuating the plunger of the syringe, is discharged through the handle and/or the housing and through the probe.

The instrument according to the disclosure can have a special connection at the proximal end of the handle for connecting to a vitrectomy device with infusion and aspiration connections by means of a hose. With such a design and the provision of a customary or coded connection, the instrument can be connected to a vitrectomy device so that it can be fed with a rinsing/cleaning fluid. It is also conceivable to aspirate using the vitrectomy device.

The handle and/or the probe can be made of plastic or metal, in particular of stainless steel. In particular, in the case of single-use devices, inexpensive manufacture from plastic is an option.

The core can also be made of plastic, wherein a core that is bent in the extended state can be a core made of plastic with shape memory. It is also conceivable to use a metal alloy with shape memory, for example a nickel-titanium shape memory alloy, as is known per se in the most varied of technical fields.

It has already been stated above that the dimensions of the probe are to be designed in such a way that the probe can be inserted into the eye through a commercially available trocar. As such, the probe may have a diameter of 0.4 to 0.8 mm, preferably around approx. 0.6 mm.

The core may have a diameter of 0.4 to 0.5 mm, preferably 0.45 mm. The cleaning material attached at the distal end of the core could have a diameter of 0.35 mm in bunched and/or compressed state, wherein the material preferably unfolds when pushing out the distal part of the core, for example by splicing three or four cloth-like and/or fiber-like wipe elements.

The instrument according to the disclosure can be introduced into the eye through a trocar placed in the pars plana, namely through a minimal incision, especially since the active or cleaning element is located in the probe in the retracted state. It's literally squeezed together there. According to the desired direction into the eye, starting from the pars plana access, the core can be extended with the active element at the end, whereby the active element is enlarged. Cleaning agents can then be added through an inner channel either from a reservoir in the handle, through a connected syringe, or through active infusion and aspiration, namely through a connection to a vitrectomy device with infusion and aspiration connections. Retracting and extending the active element and more or less strong bending of the core with the active element at the end can be done by a wide variety of sliding mechanisms on and/or in the handle.

Now, there are different possibilities of designing and further improving the teaching of the present disclosure in an advantageous manner. In this respect, reference is made to the claims dependent on claim 1 on the one hand, and on the following explanations on a preferred exemplary embodiment of the disclosure by way of the drawings on the other hand. In conjunction with the explanation of the preferred exemplary embodiment of the disclosure by way of the drawings, generally preferred designs and further improvements of the teaching are also explained. In the drawings:

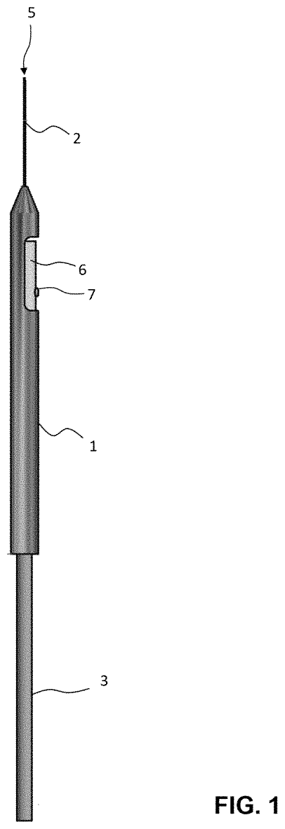

FIG. 1 is a schematic side view of an exemplary embodiment of an instrument according to the disclosure with only slightly pushed-out straight core and active element at the end,

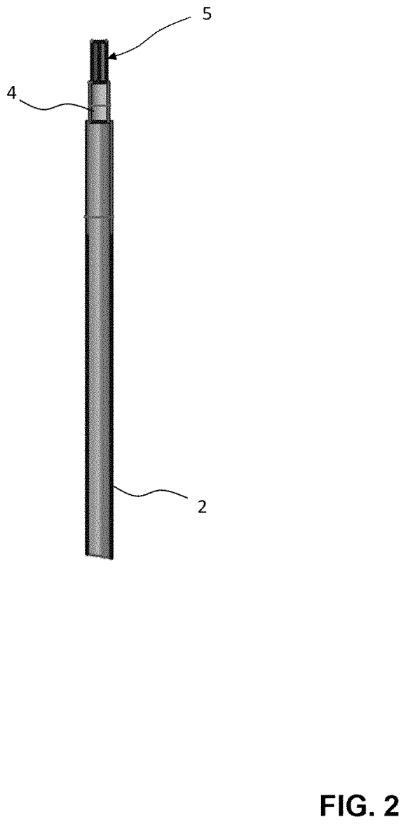

FIG. 2 is a schematic enlarged view of the probe with the core pushed out slightly and the active element at the end,

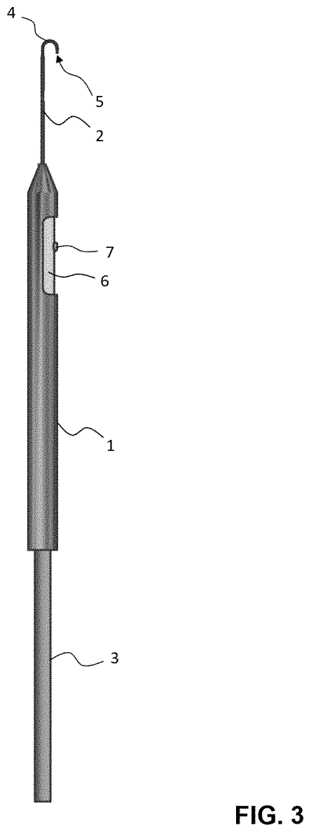

FIG. 3 is a schematic view of the instrument of FIG. 1 with the core being far out and pushed by 180 degrees and the active element at the end,

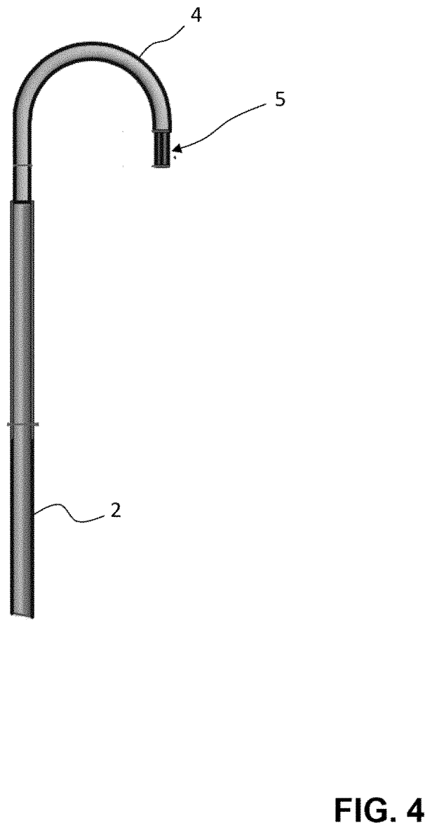

FIG. 4 is a schematic enlarged view of the probe with the core pushed out far and the active element at the end,

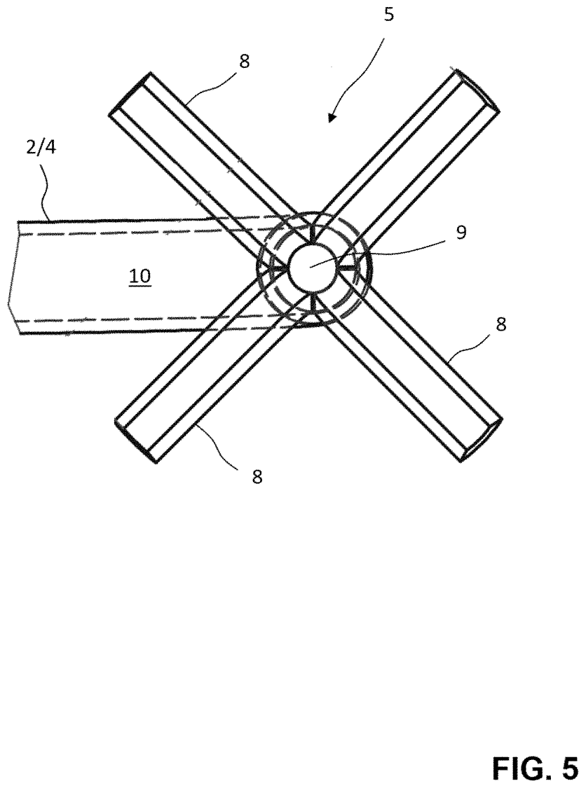

FIG. 5 is a schematic front view of the core at the end with unfolded active element with respect to the pushed-in state with an enlarged surface, and

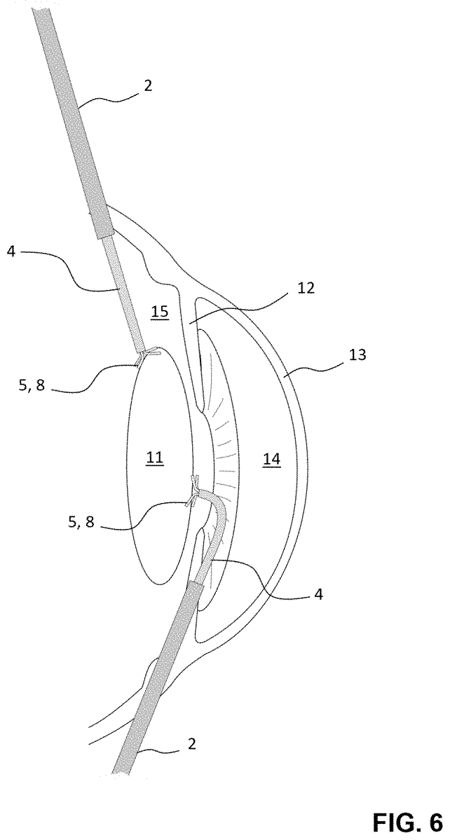

FIG. 6 is a schematic view of two engaging situations using the instrument according to the disclosure on/in the human eye.

FIG. 1 shows an embodiment of an instrument according to the disclosure for intraocular surgical procedure. The instrument comprises handle 1 and probe 2 connected to handle 1.

Rinsing and aspiration connection 3 is provided at the proximal end of handle 1.

FIG. 2 shows the object of FIG. 1 in the area of the distal end, specifically probe 2 and the slightly and/or sparsely extended core 4, which extends in a straight line out of probe 2. At the end of core 4, active element 5 is provided, as shown in detail in FIG. 5 as one of many possibilities. It consists of a cleaning material with an enlarged surface.

FIG. 3 shows the object of FIG. 1, wherein core 4 is pushed out far. Since the material of core 4 is a curved material with shape memory, core 4 bends when it is pushed out of probe 2 according to the shape memory, according to FIG. 4, by about 180 degrees. Active element 5 is arranged at the end of core 4.

At this point it should be noted that the core 4 can also be pushed out just far enough that active element 5 protrudes approximately orthogonally from the longitudinal direction of probe 2.

FIGS. 1 and 3 also show that the housing comprises an actuating device with actuating element 6. For locking the position of core 4 with respect to probe 2, locking member 7 of a locking mechanism is provided.

FIG. 5 shows the situation at the end at the distal end of probe 2 and/or core 4, according to which active element 5 is arranged at the end of core 4. In the example shown in FIG. 5, active element 5 unfolds like a mop and comprises four cleaning cloths 8 which unfold when core 4 is pushed out of probe 2. Due to their material, they are suitable for absorbing impurities. In the center of active element 5, opening 9 serving for aspiration, which opens into inner channel 10, can be provided.

Finally, FIG. 6 shows two different situations in the context of an intraocular intervention, the instrument according to the disclosure being merely indicated. Probe 2 and core 4 can be seen, wherein two different instruments or working states with a straight core 4 and a curved core 4 are shown. In both cases, active element 5 is shown at the distal end of core 4 in accordance with the above explanations. In both cases, the instrument according to the disclosure is used to clean the lens surface.

For the sake of a complete representation, lens 11, iris 12, cornea 13 as well as anterior chamber of the eye 14 and posterior chamber of the eye 15 are indicated in the schematic view according to FIG. 6.

Regarding further advantageous embodiments of the instrument according to the disclosure, reference is made to the general section of the description and the enclosed claims to avoid repetition.

Finally, it is important to note that the exemplary embodiment of the instrument according to the disclosure described above are only set forth for the purpose of discussing the teaching claimed, but do not limit it to the exemplary embodiment.

### LIST OF REFERENCE NUMERALS

1 handle

2 probe

3 rinsing and aspiration connection

4 core

5 active element

6 actuating element

7 locking device

8 cleaning cloth

9 opening

10 inner channel

11 lens

12 iris

1. An instrument for intraocular surgical procedure, comprising:
a handle;
a probe including a hollow body and connected to the handle;
a core in the probe;
an active element at a distal end of the probe for removing impurities from an eye; and
an actuating device in the handle for actuating the probe,
wherein either a) the probe is at least partially displaceable in and out of the handle and the core is firmly connected to the handle, or b) the core is at least partially slidable in and out of the handle and the probe is firmly connected to the handle, so that the core with the active element is completely within the probe or protrudes from the probe by a predetermined length.

2. The instrument according to claim 1, wherein the active element comprises at least one of a brush, mop, cloth, sponge, wiper blade, fibrous material, flat material, and a ball-like material.

3. The instrument according to claim 1, wherein the core is substantially straight when out of the handle.

4. The instrument according to claim 1, wherein the core is bent by 45° to 180° towards the distal end so that when the core is out of the handle, the core undergoes elastic relaxation and unfolds, and when the core is in the handle, the core exerts an elastic bias against an inner wall of the probe.

5. The instrument according to claim 1, wherein the handle comprises a sleeve, and an actuating element of the actuating device slides within the sleeve.

6. The instrument according to claim 5, wherein the actuating device comprises a locking mechanism for locking a relative position between the probe and the core.

7. The instrument according to claim 1, wherein the handle comprises a reservoir for a liquid which can be discharged through the probe towards the active element.

8. The instrument according to claim 1, further comprising a connection for connecting a syringe a proximal end of the handle, wherein contents of the syringe can be discharged through the handle and the probe towards the active element.

9. The instrument according to claim 1, further comprising a connection at a proximal end of the handle for connecting to a vitrectomy device having infusion and aspiration connections.

10. The instrument according to claim 1, wherein at least one of the handle and/the probe is made from plastic or metal.

11. The instrument according to claim 1, wherein the core is made from a plastic or a metal alloy having a shape memory.

12. The instrument according to claim 1, wherein the probe has dimensions that enable the probe to be inserted into an eye through a trocar having a diameter of 0.4 to 0.8 mm.
